(19) United States
(12) Patent Application Publication
(10) Pub. No.: US 2007/0240242 A1
(43) Pub. Date: Oct. 11, 2007

(54) METHOD FOR MULTIVARIATE ANALYSIS IN PREDICTING A TRAIT OF INTEREST

(75) Inventors: Steven H. Modiano, Manchester, MO (US); Dutt V. Vinjamoori, Columbia, MD (US); Pradip Das, Olivette, MO (US)

Correspondence Address: HARNESS, DICKEY & PIERCE, P.L.C., 7700 BONHOMME AVENUE, SUITE 400, ST. LOUIS, MO, 63105, US

(73) Assignee: Monsanto Technology LLC, St. Louis, MO (US)

(21) Appl. No.: 11/697,602

(22) Filed: Apr. 6, 2007

Related U.S. Application Data
(60) Provisional application No. 60/789,679, filed on Apr. 6, 2006.

(51) Int. Cl. A01H 1/00; C12N 15/82; G01N 33/48

(52) U.S. Cl. 800/284; 435/40.5

(57) ABSTRACT

A method for predicting a trait of interest in an agricultural sample comprises (a) obtaining a set of input data from: (i) at least one agronomic property; and (ii) at least one of a chemical property and physical property; (b) inputting the data into a processor containing at least one algorithm wherein the processor performs correlations of the input data with the trait of interest; and (c) outputting a predicted efficacy for the trait of interest. A computer-aided system comprises: (a) a computer readable medium including computer-executable instructions configured for estimating a trait of interest in an agricultural sample; (b) input data from: (i) at least one agronomic property; and (ii) at least one of a chemical property and physical property; and (c) an algorithm capable of correlating the data with the trait of interest; wherein the system outputs a predicted efficacy for the trait of interest. The trait of interest can include ethanol yield and/or digestibility.

### CROSS-REFERENCE TO RELATED APPLICATION

This application claims priority to U.S. Provisional Application Ser. No. 60/789,679 filed Apr. 6, 2006. The disclosure of U.S. Provisional Application Ser. No. 60/789,679 is hereby incorporated herein by reference in its entirety.

### FIELD

The present invention relates to production of cereals and livestock feeds, and also relates to production of ethanol by fermentation of starch containing plants. More specifically, the invention relates to a multivariate method for predicting a trait of interest, for example predicting high digestibility and/or predicting fermentability to yield ethanol.

### BACKGROUND

The statements in this section merely provide background information related to the present disclosure and may not constitute prior art.

Use of alternative energy sources can be desirable for several reasons, for example, reliance on fossil fuel may be decreased, and in turn air pollution may be reduced. Ethanol production by fermenting carbohydrate-containing plants is one possible source of alternative energy. For example, U.S. Pat. No. 4,568,644 to Wang et al. discusses a method for producing ethanol from biomass substrates by using a microorganism capable of converting hexose and pentose carbohydrates to ethanol, and to a lesser extent, acetic and lactic acids. U.S. Pat. No. 5,628,830 to Brink discusses a method for producing sugars and ethanol from biomass material which consists of two processes: hydrolysis of cellulose to glucose and fermentation of the glucose to ethanol.

Maximized ethanol production from biomass is economically desirable. Efforts have been made to achieve increased yield, especially by altering production processes or by adding extra steps for ethanol production. For example, U.S. Pat. No. 5,916,780 to Foody et al. discusses a process for improving economical ethanol yield by selecting feedstock with a ratio of arabinoxylan to total non-starch polysaccharides greater than about 0.39, then pretreating the feedstock to increase glucose production with less cellulose enzyme. Subsequent fermentation reportedly permits greater ethanol yield. U.S. Pat. No. 6,509,180 to Verser et al. discusses a process for producing ethanol including a combination of biochemical and synthetic conversions to achieve high yield ethanol production by preventing production of CO₂, a major limitation on the economical production of ethanol.

Maximized digestibility from biomass is also economically desirable. Grains grown and harvested for consumption by humans or by livestock have varying levels of digestibility. For livestock in particular, cost effective productivity and weight gain depends on the digestibility of the feed. The livestock feed industry has used several processing methods to improve feed value including steam flaking, reconstitution, micronisation, and high temperature, short-time extrusion. However, it would be more beneficial to predict prior to any processing step the digestibility of a particular plant variety, for example, the digestibility of a corn hybrid.

A number of techniques to characterize cellular organization of a plant are available. A plant's physical and/or chemical properties are used to analyze the plant's make-up. Chemical analysis is widely used in laboratories because it is fast and sensitive, and is suitable for automation.

Fox et al., Relations of Grain Proximate Composition and Physical Properties to Wet-Milling Characteristics of Maize, Cereal Chemistry, 69(2):191-197 (1992) discuss single factor correlations of proximate composition and physical data of maize hybrids with product yields, starch recovery data and product composition data. Fox et al. further discuss the use of multiple regression to account for additional variation in starch yield and protein content of recovered starch.

Singh et al., Compositional, Physical, and Wet-Milling Properties of Accessions Used in Germplasm Enhancement of Maize Project, Cereal Chemistry, 78(3):330-335 (2001) mention that starch yield and recovery were positively correlated with starch content and negatively correlated with protein content and absolute density. Singh et al. also mention that varieties with lower absolute densities and test weights, greater starch contents, and lower fat and protein contents would be better for wet milling than other varieties without those characteristics.

Fang et al., Neural Network Modeling of Physical Properties of Ground Wheat, Cereal Chemistry, 75(2)251-253 (1998), mention the design and training of neural network models reportedly capable of predicting physical properties of roller-milled wheat ground materials.

Gauchi and Chagnon, Comparison of Selection Methods of Explanatory Variables in PLS Regression with Application to Manufacturing Process Data, Chemometrics and Intelligent Laboratory Systems, 58:171-193 (2001) discuss selection methods of variables used in predictive models by the oil, chemical and food industries.

The industry would benefit by the availability of methods for optimizing quality, quantity and cost-of-goods for the production of ethanol through fermentation of grains and biomass. Similarly, the industry would benefit by the availability of those same methods for optimizing quality, quantity, and cost-of-goods for the production of cereals and livestock feeds that are highly digestible. In particular, new methods for determining the efficacy to yield ethanol and/or determining digestibility of individual plant varieties would represent a useful advance in the art.

### SUMMARY

The inventors have conceived of a method and system for predicting a trait of interest such as ethanol yield or digestibility in an agricultural sample. Such a method and a system for predicting ethanol yield leads to selection of preferred properties for optimum process conditions in the fermentation of grains or biomass. Such a method and a system for predicting digestibility leads to selection of preferred properties for optimum process conditions in livestock feed and cereal production.

Thus, the present disclosure provides a method for predicting a trait of interest in an agricultural sample comprising (a) obtaining a set of input data from: (i) at least one agronomic property; and (ii) at least one of a chemical property and physical property; (b) inputting the data into a processor containing at least one algorithm wherein the processor performs correlations of the input data with the trait of interest; and (c) outputting a predicted efficacy for the trait of interest.

Also provided is a computer-aided system comprising: (a) a computer readable medium including computer-executable instructions configured to estimate a trait of interest in an agricultural sample; (b) input data from: (i) at least one agronomic property; and (ii) at least one of a chemical property and physical property; and (c) an algorithm capable of correlating the data with the trait of interest; wherein the system outputs a predicted efficacy for the trait of interest.

Additional embodiments are described in the detailed description that follows.

Further areas of applicability will become apparent from the description provided herein. It should be understood that the description and specific examples are intended for purposes of illustration only and are not intended to limit the scope of the present disclosure.

### DRAWING

FIG. 1 is a block diagram of a computer system that may be used to implement a method and apparatus embodying the invention.

The drawing described herein is for illustration purposes only and is not intended to limit the scope of the present disclosure in any way.

### DETAILED DESCRIPTION

The following description is merely exemplary in nature and is not intended to limit the present disclosure, application, or uses.

The present disclosure provides a method for predicting a trait of interest in an agricultural sample comprising (a) obtaining a set of input data from: (i) at least one agronomic property; and (ii) at least one of a chemical property and physical property; (b) inputting the data into a processor containing at least one algorithm wherein the processor performs correlations of the input data with the trait of interest; and (c) outputting a predicted efficacy for the trait of interest.

Also provided is a computer-aided system comprising: (a) a computer readable medium including computer-executable instructions configured for estimating a trait of interest in an agricultural sample; (b) input data from: (i) at least one agronomic property; and (ii) at least one of a chemical property and physical property; and (c) an algorithm capable of correlating the data with the trait of interest; wherein the system outputs a predicted efficacy for the trait of interest.

A trait of interest can include any desirable trait that enhances production or marketability of a plant or plant seed. Illustrative examples include but are not limited to digestibility, fermentability to yield ethanol, quality of co-products (distillers' dried grains with or without solubles), quality of dry milled products (corn flour, corn grits, ready-to-eat cereals, brewing adjuncts, extruded and sheeted snacks, breadings, batters, prepared mixes, fortified foods, animal feeds, hominy, corn gluten feed, etc.), quality of industrial products, etc.

A property as used herein is something measured or evaluated in an agricultural sample, for example, a sample obtained from the plant, or a group of plants such as a crop plant or hybrid.

As used herein, the phrase agricultural sample can be any plant of interest, including an individual plant, more than one plant, a plant variety or hybrid, a crop breed, or crop variety. Typically, the plant is a cereal variety such as, for example, maize, wheat, barley, rice, rye, oat, sorghum, or soybean. Particularly for measuring agronomic properties or physical properties of a plant, the step of obtaining a sample from the plant can include obtaining one or more seeds or grains from a plant, or, obtaining whole plant samples from, for example, a field. Obtaining a sample, in some embodiments, can merely be the identification of one or more plants on which measurements will be made.

An agricultural sample can include one or more seeds from the plant. Any seed can be utilized in a method or assay of the invention. Individual seeds or seeds in a batch can be analyzed.

An agricultural sample can include other plant tissues. As used herein, plant tissues include but are not limited to, any plant part such as leaf, flower, root, and petal.

As used herein, input data is any data obtained by measuring at least one property. Obtaining a set of input data from the above-listed properties can include obtaining the data from a database, and can also include measuring the value of an agronomic property, a chemical property, and/or a physical property. The values can be actual values, or can be assigned numbers related to the absolute value. Any combination of data can be obtained including, for example, agronomic data and chemical data, agronomic data and physical data, or each of agronomic data, physical data, and chemical data.

A user of the methods and systems (including servers, computers, etc.) can include an individual, a corporation, a partnership, a government agency, a research institution or any other person or entity that has an interest in or need for information regarding a trait of interest such as ethanol yield or digestibility of a crop plant or various other plants. Non-limiting examples include farmers, seed distributors, buyers, and processors.

Screening hybrids for a trait of interest typically precedes processing of the grain by milling, cooking, etc., and can start with measuring at least one agronomic property, and further includes screening hybrids from a mixture by measuring at least one of a chemical property and a physical property in a plant. If the trait of interest is ethanol yield, by taking into account agronomic properties, the efficacy of ethanol yield can be predicted, for example, according to the yield per acre. By additionally measuring at least one of a chemical or physical property, fermentability to yield ethanol is included as a factor which contributes to the efficacy of ethanol yield. High and low ethanol yield varieties have distinguishable characteristics in chemical and physical properties as do high and low digestibility hybrids, and identification of these characteristics leads to predicting and screening a plant for the trait of interest. Measuring can include, for example, assessing a chemical profile for particular plant hybrids, studying the subcellular organization of endosperm cells of high and low ethanol-yield hybrids or high and low digestibility hybrids, and/or assessing agronomic characteristics for hybrids of interest.

To select a plant variety preferable for a particular trait of interest, a method for the present invention involves the application of a destructive or non-destructive technique or a combination thereof for the generation of agronomic, chemical, kinetic, physical, rheological, and morphological data for a representative population with a wide range of variation.

The step of obtaining a set of input data from at least one of a chemical property and physical property can include obtaining any acceptable plant tissue conducive to measuring the particular property, including, for example, foliage, seed, seed part, root, etc. In some embodiments, a seed is obtained from the plant. In a further embodiment, endosperm is obtained from the seed and the measurement is done with the endosperm sample.

In a still further embodiment, obtaining a set of input data includes obtaining at least one of sectioned (thin, flat slices) and grind (scratched with a razor blade to form powder or grinding in a mechanical grinder) samples.

More than one set of data from one plant variety can be obtained for each property to ensure accuracy of the analysis. If two or more plants are analyzed, samples from each plant should generally be obtained from the same tissue.

Agronomic Properties

As used herein, an agronomic property is any property relating to the science of crop production including crop yield, seed vigor, relative maturity, pest resistance, seed handling, etc. Relative maturity as used herein is the cessation of dry weight accumulation by the kernel, and, therefore, maximum yield. Seed handling as used herein includes packing density, fragility, moisture content, threshability, etc.

Other agronomic properties include days to heading, plant height, lodging resistance, emergence vigor, vegetative vigor, porosity, stress tolerance, disease resistance, branching, flowering, seed set, and standability.

Obtaining a set of input data from at least one agronomic property includes measuring the value of an agronomic property. Agronomic data has already been obtained and is available in the industry for many crops, as this same data is used to compare desired characteristics when determining which crop seed to plant. An ordinarily skilled artisan can obtain agronomic data for practicing this invention from an already existing database. Agronomic data can also be obtained by taking appropriate field measurements to determine, for example, crop yield, seed vigor, etc.

It is important to include agronomic data in the analysis of predicting ethanol yield for a crop as a whole for several reasons. Agronomic data takes into account the overall productiveness of a crop plant or a hybrid. For example, if hybrid A produces more ethanol per bushel than does hybrid B, one would believe that hybrid A is the choice crop. However, if hybrid A is particularly susceptible to common pests, or typically is a low yielding crop, it can be more beneficial to choose hybrid B. Agronomic data also takes into account factors such as timing of harvest and/or cost to optimize ethanol yield.

It is also important to include agronomic data in the analysis of predicting digestibility for a crop as a whole as discussed with respect to fermentability. Relative maturity, kernel hardness, timing of harvest, and other factors can be indicators of digestibility and can be considered when optimizing digestibility of a plant.

Chemical Properties and Physical Properties

A characteristic, highly organized, protein matrix consisting of numerous, tightly packed protein bodies, pressed against amyloplasts, is present in the endosperm cells of a low ethanol yield plant. Plants with such characteristics have cells that are more difficult to break apart and release cell contents, as single, protein-free starch grains. While not bound by theory, it is believed that the ability to resist breaking apart, or a greater degree of starch-protein association, can be a major limitation on the economic production of ethanol from plant sources since the availability of starch grains is reduced. As used herein, the phrase “degree of starch-protein association” indicates the level to which starch and protein are connected to each other as determined by, for example, the methods described below. In the process of digestion and fermentation, starch grains are broken down to simple sugars, typically by alpha amylase and/or gluco amylase. Ethanol is produced when yeast feed on the sugars.

Measurements of the value of physical and chemical properties as described herein can be useful input data in predicting digestibility or ethanol yield. Physical and chemical properties described below are indicators of digestibility or fermentability, and the degree of fermentability is a factor in determining efficacy to yield ethanol.

A higher concentration of a certain substance can reveal information regarding a trait of interest in an agricultural sample. Thus, measuring chemical properties of a plant can be carried out through profiling a certain substance in cells or tissues taken from the plant. A wide variety of substances can be evaluated for the purpose of screening plants and plant varieties. Generally, a substance to be measured will be selected based upon species of the plant to be analyzed. At least one substance needs to be measured and an ordinarily skilled artisan can determine optimal or preferable number of target substances based on the plant to be used. Typically, a substance to be measured is selected from protein, starch, and lipid.

The chemical property can be selected from oil content, fiber content, moisture content, amino acid content, protein content or starch content. Oil content can include both the amount and type of oil. Fiber content can include both the amount and classification of fiber. Amino acid content can include both the amount and type of amino acid. Protein content can include both amount and type of protein. Starch content can include both amount and classification of starch.

The inventors have determined that plant's chemical properties, assessed using chromatographic analyses, show distinctly different protein elution profile for high and low fermentable plant lines. In particular, for example, specific plant proteins such as zeins are more abundant in low fermentable corn lines in comparison with high fermentable corn lines. Zein proteins are hydrophobic and are found bound to starch through non-covalent bonding and hydrophobic interactions. Accordingly, higher zein content can play an important role in the fermentation yield process such as inhibiting the fermentation process by limiting the starch availability. Zein proteins contain higher amounts of thiols and disulfides relative to other proteins, thus, in one embodiment, quantification of thiols and disulfides in a protein sample is an indicator of the amount of zein protein.

Similarly, plants' chemical properties show distinctly different protein elution profiles for high and low digestibility plant lines. Zein proteins are more abundant in low digestibility corn lines and less abundant in high digestibility corn lines.

Any chemical analysis technique known in the art can be used for the determination of chemical properties, such as determination of protein, starch and lipid compositions. Among various chemical analysis techniques, separation techniques are generally desirable for an application of the present invention. Examples of chemical analysis techniques include, but are not limited to, HPLC, MALDI-TOF MS, capillary electrophoresis, RP-HPLC on-line MS, gel electrophoresis, SDS page, 2-D gel electrophoresis, and combinations thereof.

In one embodiment, a method for predicting a trait of interest includes obtaining a set of input data from a high-throughput method employing a high-throughput analyzer capable of producing results quickly. The input data can be obtained from at least one of a chemical property and physical property, but ideally provides data on more than one property in a short period of time. Fast delivery of the result can help in optimizing digestibility or ethanol yield at a plant level. Illustrative analyzers include but are not limited to, for example, HPLC, MALDI-TOF MS, capillary electrophoresis, RP-HPLC on-line MS, gel electrophoresis and combinations thereof.

In some embodiments, the input data is obtained for the chemical profile of target substances such as protein, starch or lipid. In particular embodiments, the protein is zein which comprises α-zein, β-zein and γ-zein proteins. In other embodiments, a chemical property is measured to determine sulfur content, an indicator of thiol and disulfide containing proteins.

A trait of interest can also be predicted by measuring the physical properties of a plant indicative of the trait of interest. In one embodiment, the method comprises determining the starch density of a sample of the plant in suspension. Starch density is the amount of starch visualized or measured in some discrete unit, for example, a volume or an area of an image. In some embodiments, the method comprises measuring protein through immunoprecipitation or immunostaining. In other embodiments, the method comprises staining the sample with a stain reagent for protein, lipid, lipoprotein or carbohydrate, presenting an image of the stained sample and determining starch-protein association by analyzing the image.

The physical property can be selected from non-cellular properties including absolute seed density, seed test weight, seed hardness, seed size, hard to soft endosperm ratio, germ size, color, cracking, water uptake, pericarp thickness, or crown size.

In a particular embodiment, visualizing the cellular characteristic includes (a) staining the sample with a stain reagent for at least one of protein, lipid, lipoprotein, and carbohydrate; (b) presenting an image of the stained sample; and (c) measuring the cellular characteristic including analyzing the presented image.

A study of physical properties of plants with microtechniques reveals that each of high-ethanol and low ethanol yield plants has distinguishable cellular characteristics as do high and low digestibility plants. No significant differences are found between starch grains of high-ethanol and low ethanol-yield hybrids in terms of size, shape, indices of refraction, ratios of starch grain populations, and color of staining. However, in samples of high-ethanol-yield hybrids, starch grains are randomly dispersed inside the cell, easy to isolate, thus forming suspensions containing higher densities of starch grains. In such high-ethanol-yield samples, starch grains are generally dispersed in suspension as single structures, rarely associated with protein, whereas, for samples of low ethanol yield hybrids, starch grains are highly organized inside the cell, difficult to isolate, thus resulting in low-density-starch grain suspensions. These low-density-starch grains are frequently present in suspension as aggregates or clusters, and are frequently associated with protein. Specifically, microscopic examination shows that the starch grains of high-ethanol-yield hybrids are loosely packed inside the cells and rarely show irregular surfaces. Starch grains of low ethanol-yield hybrids are tightly packed against each other, and show materials associated with/or on the amyloplast surface. These same findings apply to other traits of interest including digestibility.

Protein staining shows significant differences between high-ethanol and low ethanol yield hybrids: the protein matrix of high-ethanol-yield samples is smooth, continuous, and fragile, but the protein matrix of low ethanol-yield samples is irregular, thicker, with a high density of globular structures. Therefore, the grains dispersed in aggregates or clusters and associated with proteins can be evaluated as low ethanol-yield variety. The findings are similar for high and low digestibility hybrids.

The phrase “protein packing” as used herein describes the visualization of the protein matrix. In some embodiments, visualization of protein packing is used to analyze starch-protein association. The degree of protein packing can be measured in any manner known in the art or described herein, or relative values can be assigned to represent varying degrees of protein packing. In this manner, data obtained from measuring protein packing is useful as input data.

The phrase “starch protein matrix” as used herein refers to the association of starch with surrounding protein matrices, usually in endosperm cells.

Protein packing, the starch protein matrix, and starch density are cellular characteristics, any one of which can be measured in a given plant sample. Typically, these properties are measured in endosperm samples.

Visualization of cell components generally requires sample preparation as an initial step. Samples for microscopic analysis can be taken from any part of the plant of interest. Generally, it is desirable to obtain samples from plant parts being a major starch source. Illustratively, endosperm tissues can be used for sample preparation.

After samples are taken from the plants, they can be stained for better microscopic observation. Staining targets can be changed depending upon the plant to be used in production of the trait of interest. The targets are generally selected from protein, lipid, lipoprotein, and carbohydrate. Staining procedures are well known in the art and practically any known procedure can be successfully employed for the present invention. A specific staining procedure will be suitably selected in accordance with the staining target. Like staining protocols, any known staining reagent can be used for the present invention. Illustratively, mercurochrome, iodine and Sudan IV can be used for protein, starch and lipid staining, respectively. However, the choice of reagents is not necessarily determinative for the outcome of the invention. Samples can be stained with one or more reagents. For example, a sample can be stained with mercurochrome to identify proteins containing thiols and disulfides, and then counterstained with acridine orange to identify amyloplasts. Double-staining in this manner allows visualization of co-localized targets.

To visualize cellular characteristics of a sample, an image of the stained sample is presented. Typically, microscopy techniques can be employed. Any known microscopy technique such as, for example, light, confocal, hyperspectral, and electron microscopy, can be used to determine subcellular organization of cells or tissues of the sample plants. An ordinarily skilled artisan can choose suitable microscopes in accordance with samples used in the method. Examples of microscopes for such techniques include, but are not limited to, differential interference contrast (DIC) microscope, polarized light microscope, fluorescence microscope, epi-fluorescence microscope, confocal microscope, hyperspectral microscope, scanning electron microscope (SEM), and transmission electron microscope (TEM).

Visualizing the cellular characteristics by microscope enables measurement of the cellular organization of the samples. For example, the respective amounts of starch grains associated with protein and without protein present in the plant samples can be determined by counting of associated grains. This can serve as basis for determining high-ethanol and low ethanol yield traits. Observation and counting can be conducted in various fashions such as direct observation through an eyepiece and examination of pictures taken through a microscope. Starch-protein association can be determined by quantification of fluorescence, fluorescent dots, determination of fluorescence intensity, or determination of area of fluorescence. Analysis of subcellular organization, such as counting of grains, can be automated with the assistance of a computer device or software, or combination of both computer device and software.

Other visualizing techniques can be employed to analyze a plant's physical characteristics, including but not limited to fluorescent plate readers, spectrophotometer, light scatter, hyperspectral technologies, fluorimeters, flow cytometers, NIR spectroscopy, and Raman spectroscopy.

Thus, data obtained from a database and/or the above-described techniques is inputted into a processor. The processor contains at least one algorithm and performs correlations of the input data with the trait of interest. Processors are generally known in the art, but can be such as described below. A suitable algorithm can be one that correlates the input data with the trait of interest, and is also described in the computer-aided system below.

The outcome of processor's correlating is the output of a predicted efficacy for a trait of interest, a function of both agronomic properties and chemical and/or physical properties. Outputting a predicted efficacy includes rating of the input data for ability to predict efficacy.

Computer-Aided System

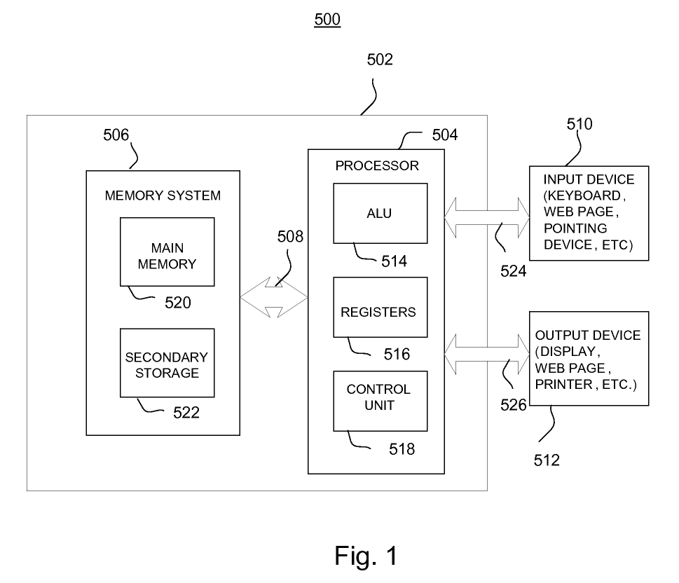

According to some embodiments, input data or a set of input data, obtained as described above, is introduced into a computer-aided system and subjected to analysis in the system exemplified in FIG. 1. Using the input data, a predicted efficacy for a trait of interest is computed using an algorithm that takes into account the values measured. The algorithm can include the input data for all the properties or a selection of the properties. The output data, as described below, is a predicted efficacy for a trait of interest.

Referring to FIG. 1, an operating environment for an illustrated embodiment of the present invention is a computer-aided system 500 with a computer 502 that comprises at least one processor 504, in conjunction with a memory system 506 interconnected with at least one bus structure 508, an input device 510, and an output device 512.

The illustrated processor 504 is of familiar design and includes an arithmetic logic unit (ALU) 514 for performing computations, a collection of registers 516 for temporary storage of data and instructions, and a control unit 518 for controlling operation of the system 500. Any of a variety of processors, including at least those from Digital Equipment, Sun, MIPS, Motorola, NEC, Intel, Cyrix, AMD, HP, and Nexgen, are equally preferred for the processor X. The illustrated embodiment of the invention operates on an operating system designed to be portable to any of these processing platforms.

The memory system 506 generally includes high-speed main memory 520 in the form of a medium such as random access memory (RAM) and read only memory (ROM) semiconductor devices, and secondary storage 522 in the form of long term storage mediums such as floppy disks, hard disks, tape, CD-ROM, flash memory, etc. and other devices that store data using electrical, magnetic, optical or other recording media. The main memory 520 also can include video display memory for displaying images through a display device. Those skilled in the art will recognize that the memory system 506 can comprise a variety of alternative components having a variety of storage capacities.

The input device 510 and output device 512 are also familiar. The input device 510 can comprise a keyboard, a mouse, a physical transducer (e.g. a microphone), etc. and is interconnected to the computer 502 via an input interface 524. The output device 512 can comprise a display, a printer, a transducer (e.g. a speaker), etc, and be interconnected to the computer 502 via an output interface 526. Some devices, such as a network adapter or a modem, can be used as input and/or output devices.

As is familiar to those skilled in the art, the computer system 500 further includes an operating system and at least one application program. Both are resident in the illustrated memory system 506. The operating system is the set of software which controls the computer system operation and the allocation of resources. The application program is the set of software that performs a task desired by the user, using computer resources made available through the operating system. The application program contains an algorithm, a function for solving problems. The algorithm can be used to determine correlations, and as such, can correlate the input data with the trait of interest, for example, digestibility or efficacy to yield ethanol. Illustratively, for a given data set entered through the input device 510, an algorithm capable of correlating the input data with the trait of interest or the processor 504 comprising the algorithm transforms individual data points into a single value indicative of the trait of interest of the plant or variety from which the data set was obtained.

According to the embodiments of this invention, a correlation is the establishment of a relationship between random variables. As demonstrated above, an algorithm can be used to determine correlations, correlating the input data with a trait of interest.

In some embodiments, the correlation is a direct indicator or an indirect indicator of the predicted trait.

In other embodiments, determining a correlation includes comparing at least one measured property to a predetermined threshold property.

Ideally, an exemplary system according to the invention may track large numbers of variables to identify hybrids or plant species with a trait of interest. The system can utilize statistical formulae to identify hybrids with high digestibility, high fermentability, high efficacy to yield ethanol.

Thus, in some embodiments, an algorithm includes a multivariate data analysis of the input data. Multivariate analysis as used herein refers to any statistical technique used to analyze data that arises from more than one variable.

Illustratively, the multivariate data analysis is selected from at least one of the group consisting of principal component analysis, principal component regression, factor analysis, partial least squares, fuzzy clustering, artificial neural networks, parallel factor analysis, Tucker models, generalized rank annihilation method, locally weighted regression, ridge regression, total least squares, principal covariates regression, Kohonen networks, linear or quadratic discriminant analysis, k-nearest neighbors based on rank-reduced distances, multilinear regression methods, soft independent modeling of class analogies, and robustified versions of the above obvious non-linear versions.

In accordance with the practices of persons skilled in the art of computer programming, the present invention is described with reference to symbolic representations of operations that are performed by the computer system 500. Such operations are referred to as being computer-executed or computer-executable. It will be appreciated that the operations which are symbolically represented include the manipulation by the processor 504 of electrical signals representing data bits and the maintenance of data bits at memory locations in the memory system 506, as well as other processing of signals. The memory locations where data bits are maintained are physical locations that have particular electrical, magnetic or optical properties corresponding to the data bits. The invention can be implemented in a program or programs, comprising a series of instructions stored on a computer-readable medium. The computer-readable medium can be any media capable of use by a computer, including any of the devices, or a combination of the devices, described above in connection with the memory system 506.

After performing the correlation, the system or method produces an output of predicted efficacy for the trait of interest. As discussed above, the predicted efficacy for the trait of interest is a function of both agronomic properties and chemical and/or physical properties.

In some embodiments, the output includes a rating of more than one measured property for ability to predict efficacy, where the rating is a function of the trait of interest associated with the plant. In still other embodiments, the system outputs a predicted efficacy and further rates the properties for ability to predict efficacy.

The present system and methods also enable ready comparisons between target populations where a predicted efficacy for the trait of interest is unknown with control groups.

When introducing elements or features and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

The description of the disclosure is merely exemplary in nature and, thus, variations that do not depart from the gist of the disclosure are intended to be within the scope of the disclosure. Such variations are not to be regarded as a departure from the spirit and scope of the disclosure.

What is claimed is:

1. A method for predicting a trait of interest in an agricultural sample, the method comprising:
(a) obtaining a set of input data from:
(i) at least one agronomic property; and
(ii) at least one of a chemical property and physical property;
(b) inputting the data into a processor containing at least one algorithm wherein the processor performs correlations of the input data with the trait of interest; and
(c) outputting a predicted efficacy for the trait of interest.

2. The method of claim 1, wherein the trait of interest is ethanol yield.

3. The method of claim 1, wherein the trait of interest is digestibility.

4. The method of claim 1, wherein obtaining the set of input data from (i) at least one agronomic property and (ii) at least one of a chemical property and physical property comprises obtaining the data from a database.

5. The method of claim 1, wherein obtaining a set of input data from at least one agronomic property includes measuring the value of an agronomic property selected from the group consisting of crop yield, seed vigor, relative maturity, pest resistance, seed handling, days to heading, plant height, lodging resistance, emergence vigor, vegetative vigor, porosity, stress tolerance, disease resistance, branching, flowering, seed set, and standability.

6. The method of claim 1, wherein the obtaining a set of input data includes obtaining a sample from a plant.

7. The method of claim 6, wherein the plant is selected from the group consisting of maize, wheat, barley, rice, rye, oat, sorghum, and soybean.

8. The method of claim 6, wherein obtaining the sample includes obtaining a sample from endosperm associated with the plant.

9. The method of claim 6, wherein obtaining a set of input data includes measuring the value of a chemical property selected from the group consisting of oil content, fiber content, moisture content, amino acid content, protein content, and starch content.

10. The method of claim 9, wherein measuring the value of the chemical property includes measuring protein content, and wherein the protein content comprises at least one zein protein selected from the group consisting of α-zein protein, β-zein protein, and γ-zein protein.

11. The method of claim 8, wherein obtaining a set of input data includes measuring the value of a chemical property comprising measuring a sulfur content.

12. The method of claim 6, wherein obtaining a set of input data includes measuring the value of a chemical property using a separation technique selected from the group consisting of HPLC, MALDI-TOF MS, capillary electrophoresis, RP-HPLC on-line MS, gel electrophoresis, SDS page, two-dimensional gel electrophoresis, and combinations thereof.

13. The method of claim 6, wherein obtaining a set of input data includes measuring the value of a physical property including at least one of a non-cellular characteristic and a cellular characteristic of the sample.

14. The method of claim 6, wherein obtaining a set of input data includes measuring the value of a physical property including a non-cellular characteristic selected from the group consisting of absolute seed density, seed test weight, seed hardness, seed size, hard to soft endosperm ratio, germ size, color, cracking, water uptake, pericarp thickness, and crown size.

15. The method of claim 6, wherein obtaining a set of input data includes measuring the value of a physical property including visualizing a cellular characteristic of the sample.

16. The method of claim 15, wherein the cellular characteristic is at least one of protein packing, starch protein matrix and starch density.

17. The method of claim 15, wherein visualizing the cellular characteristic includes analyzing the sample by at least one of immunostaining and immunoprecipitation.

18. The method of claim 15, wherein visualizing the cellular characteristic includes:
(a) staining the sample with a stain reagent for at least one of protein, lipid, lipoprotein, and carbohydrate;
(b) presenting an image of the stained sample; and
(c) measuring the cellular characteristic including analyzing the presented image.

19. The method of claim 18, wherein staining includes staining with at least one stain reagent selected from the group consisting of mercurochrome, Sudan IV, and iodine.

20. The method of claim 18, wherein presenting an image includes obtaining an image with a microscope selected from the group consisting of differential interference contrast (DIC) microscope, light microscope, polarized light microscope, fluorescence microscope, epi-fluorescence microscope, confocal microscope, hyperspectral microscope, scanning electron microscope (SEM), and transmission electron microscope (TEM).

21. The method of claim 18, wherein analyzing the image includes quantification of fluorescent dots, determination of fluorescence, fluorescence intensity, or determination of area of fluorescence.

22. The method of claim 18, wherein analyzing the image includes analyzing the image using computer software.

23. The method of claim 1, wherein the outputting a predicted efficacy includes a rating of the input data for ability to predict efficacy.

24. A computer-aided system comprising:
(a) a computer readable medium including computer-executable instructions configured to estimate a trait of interest in an agricultural sample;
(b) input data from:
(i) at least one agronomic property; and
(ii) at least one of a chemical property and physical property; and
(c) an algorithm capable of correlating the data with the trait of interest; wherein the system outputs a predicted efficacy for the trait of interest.

25. The system of claim 24, wherein the trait of interest is ethanol yield.

26. The system of claim 24, wherein the trait of interest is digestibility.

27. The system of claim 24, wherein the input data from: (i) at least one agronomic property and (ii) at least one of a chemical property and physical property is obtained from a database.

28. The system of claim 24, wherein the input data from at least one agronomic property includes crop yield, seed vigor, relative maturity, pest resistance, seed handling, days to heading, plant height, lodging resistance, emergence vigor, vegetative vigor, porosity, stress tolerance, disease resistance, branching, flowering, seed set, and standability.

29. The system of claim 24, wherein the input data from at least one of a chemical property and physical property is obtained from a plant.

30. The system of claim 29, wherein the plant is selected from the group consisting of maize, wheat, barley, rice, rye, oat, sorghum, and soybean.

31. The system of claim 24, wherein the input data includes data from at least one chemical property selected from the group consisting of oil content, fiber content, moisture content, amino acid content, protein content, and starch content.

32. The system of claim 24, wherein the input data includes data from at least one physical property selected from at least one of a non-cellular characteristic or a cellular characteristic of a plant.

33. The system of claim 24, wherein the system further comprises a user interface for interfacing the computer-aided system.

34. The system of claim 24, wherein the algorithm includes multivariate data analysis selected from at least one of the group consisting of principal component analysis, principal component regression, factor analysis, partial least squares, fuzzy clustering, artificial neural networks, parallel factor analysis, Tucker models, generalized rank annihilation method, locally weighted regression, ridge regression, total least squares, principal covariates regression, Kohonen networks, linear or quadratic discriminant analysis, k-nearest neighbours based on rank-reduced distances, multilinear regression methods, soft independent modeling of class analogies, and robustified versions of the above obvious non-linear versions.

35. The system of claim 24, wherein the system outputs a predicted efficacy and further rates the input data for ability to predict efficacy.
